(19) United States
(12) Patent Application Publication
(10) Pub. No.: US 2022/0323028 A1
(43) Pub. Date: Oct. 13, 2022

(54) PACKAGING FOR CT SCANNER FOR SPECTRAL IMAGING

(71) Applicant: Minfound Medical Systems Co. Ltd., Hangzhou (CN)

(72) Inventor: Abdelaziz Ikhlef, Hudson, OH (US)

(21) Appl. No.: 17/716,616

(22) Filed: Apr. 8, 2022

Related U.S. Application Data
(60) Provisional application No. 63/173,563, filed on Apr. 12, 2021.

(51) Int. Cl. A61B 6/00; A61B 6/03

(52) U.S. Cl. CPC A61B 6/4429; A61B 6/4233; A61B 6/4275; A61B 6/4035; A61B 6/032

(57) ABSTRACT

A CT detector module may include a module frame, a first rigid flex board, a main routing substrate arranged on the first rigid flex board, a high-density scintillator-photodiode array arranged on and electrically connected to the main routing substrate, and a low-density scintillator-photodiode array electrically connected to the main routing substrate. The first rigid flex board may include a central portion, a first lateral portion, a second lateral portion, a first flexible portion extending between and connecting the central portion and the first lateral portion, and a second flexible portion extending between and connecting the central portion and the second lateral portion. The central portion may be arranged on a first surface of the mounting frame. The first lateral portion may be disposed on a second surface of the mounting frame. The second lateral portion may be disposed on a third surface of the mounting frame.

### CROSS-REFERENCE TO RELATED APPLICATIONS

This application claims priority to U.S. Provisional Patent Application No. 63/173,563, filed on Apr. 12, 2021, the contents of which are incorporated by reference herein in its entirety.

### TECHNICAL FIELD

This disclosure relates generally to diagnostic imaging and, more particularly, to an apparatus for a computed tomography (CT) scanner, such as a dual layer detector module for spectral CT.

### BACKGROUND

Typically, in CT imaging systems, a rotatable gantry includes an x-ray tube, detector, data acquisition system (DAS), and other components that rotate about a patient that is positioned at the approximate rotational center of the gantry. X-rays emitted from the x-ray tube, are attenuated by the patient, and are received at the detector. The detector typically includes a photodiode-scintillator array of pixelated elements that convert the attenuated x-rays into photons within the scintillator, and then to electrical signals within the photodiode array. The electrical signals are digitized and then received within the DAS, processed, and the processed signals are transmitted via a slipring (from the rotational side to the stationary side) to a computer or data processor for image reconstruction, where an image is formed.

The gantry typically includes a pre-patient collimator that defines or shapes the x-ray beam emitted from the x-ray tube. X-rays passing through the patient can cause x-ray scatter to occur, which can cause image artifacts. Thus, x-ray detectors typically include an anti-scatter grid (ASG) for collimating x-rays received at the detector.

Third generation multi-slices CT scanners typically include detectors having scintillator/photodiodes arrays. These detectors are positioned in an arc where the focal spot is the center of the corresponding circle. These detectors generally have scintillation crystal/photodiode arrays, where the scintillation crystal absorbs x-rays and converts the absorbed energy into visible light. A photodiode is used to convert the light to an electric current. The reading is typically linear to the total energy absorbed in the scintillator.

Typically, CT systems obtain raw data and then reconstruct images using various known pre-processing and post-processing steps to generate a final reconstructed image. That is, CT systems may be calibrated to account for x-ray source spectral properties, detector response, and other features, to include temperature. Raw x-ray data are pre-processed using known steps that include offset correction, reference normalization, and air calibration steps, as examples.

In recent years, the development of volumetric or cone-beam CT technology has led to an increase in the number of slices used in CT detectors for computed tomography systems. The detector technology used in large coverage CT enables greater coverage in patient scanning by increasing the area exposed, by using back-illuminated photodiodes. A typical detector includes an array of 16, 32, or 64 slices. However, the need for cardiac imaging has become of greater interest to enable imaging of the heart within one rotation of the detector, substantially increasing the width of the detector in the Z-axis (e.g., along the patient length), leading to a detector having 256 or more slices. Because it is impractical to build very large modules in monolithic structure to cover this number of slices and this width in the Z-axis, due to manufacturing cost and reliability concerns, smaller modules (mini-modules) are built along the Z-axis and placed along the Z-axis to build the overall length of 256 or more slices.

In the last decade, spectral computed tomography (SCT) has been of particular interest. This technology enables the same object to be measured with different energy spectra and spectral weightings. SCT may be used to differentiate and classify material composition, by using attenuation values acquired with different energy spectra. The measurements with different spectra may be obtained in a variety of ways, for example, via a) a dual layer detector, b) fast KV switching between 80 and 140 kV from view to view, c) two KV spectra with different x-ray filter, and/or d) two x-ray sources configured to emit x-rays perpendicularly relative to one another (e.g., at 90 degrees). Each method of obtaining these measurements has advantages and disadvantages, which may include a balancing of costs and performance. In this disclosure, we describe several concepts to achieve a dual layer detector design, with appropriate electronic attachments and connections.

### SUMMARY

In examples, CT detector module may include a module frame, a first rigid flex board, a main routing substrate arranged on the first rigid flex board, a high-density scintillator-photodiode array arranged on and electrically connected to the main routing substrate, and a low-density scintillator-photodiode array electrically connected to the main routing substrate. The first rigid flex board may include a central portion, a first lateral portion, a second lateral portion, a first flexible portion extending between and connecting the central portion and the first lateral portion, and a second flexible portion extending between and connecting the central portion and the second lateral portion. The central portion may be arranged on a first surface of the mounting frame. The first lateral portion may be disposed on a second surface of the mounting frame. The second lateral portion may be disposed on a third surface of the mounting frame.

The foregoing and other potential aspects, features, details, utilities, and/or advantages of examples/embodiments of the present disclosure will be apparent from reading the following description, and from reviewing the accompanying drawings.

### BRIEF DESCRIPTION OF THE DRAWINGS

While the claims are not limited to a specific illustration, an appreciation of various aspects may be gained through a discussion of various examples. The drawings are not necessarily to scale, and certain features may be exaggerated or hidden to better illustrate and explain an innovative aspect of an example. Further, the exemplary illustrations described herein are not exhaustive or otherwise limiting, and embodiments are not restricted to the precise form and configuration shown in the drawings or disclosed in the following detailed description. Exemplary illustrations are described in detail by referring to the drawings as follows:

FIG. 1 is a perspective view of a CT imaging system.

FIG. 2 is a planar cross-section of the system illustrated in FIG. 1.

FIG. 3 is an example of an imaging chain.

FIG. 4A illustrates a perspective view of an exemplary dual layer detector module.

FIG. 4B illustrates a side view of the exemplary dual layer detector module of FIG. 4A.

FIG. 4C illustrates an exploded view of the exemplary dual layer detector module of FIG. 4A.

FIGS. 4D and 4E illustrates a close-up side view and a simplified schematic representation, respectively, of a portion of the exemplary dual layer detector module of FIG. 4B.

FIGS. 5A and 5B illustrate a side view and a simplified schematic representation, respectively, of a portion of an exemplary dual layer detector module.

FIGS. 6A and 6B illustrate a side view and a simplified schematic representation, respectively, of a portion of an exemplary dual layer detector module.

FIGS. 7A and 7B illustrate a side view and a simplified schematic representation, respectively, of a portion of an exemplary dual layer detector module.

FIGS. 8A and 8B illustrate a side view and a simplified schematic representation, respectively, of a portion of an exemplary dual layer detector module.

### DETAILED DESCRIPTION

Reference will now be made in detail to embodiments of the present disclosure, examples of which are described herein and illustrated in the accompanying drawings. While the present disclosure will be described in conjunction with embodiments and/or examples, they do not limit the present disclosure to these embodiments and/or examples. On the contrary, the present disclosure covers alternatives, modifications, and equivalents.

The operating environment of disclosed embodiments is described with respect to a multi-slice computed tomography (CT) system. Embodiments are described with respect to a “third generation” CT scanner, however it is contemplated that the disclosed embodiments are applicable to other imaging systems as well.

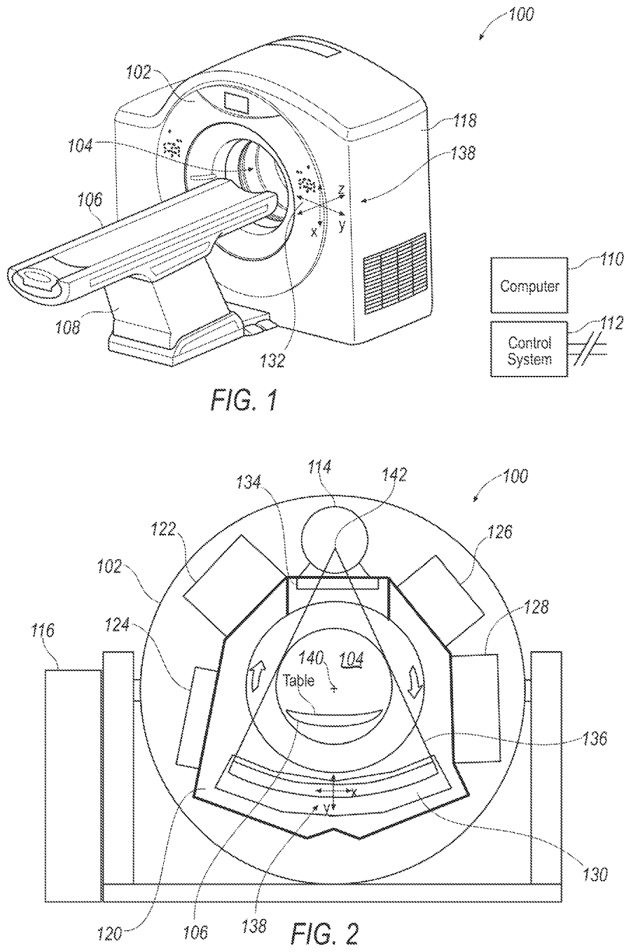

Referring to FIGS. 1 and 2, a computed tomography (CT) system 100 includes a gantry 102 having an opening 104. A patient table 106 is positioned on a support structure 108, and patient table 106 is axially controllable such that a patient (not shown) positioned on table 106 may be positioned within opening 104. A computer system 110 provides operator instructions and other control instructions to a control system 112. Computer system 110 also may include image reconstruction algorithms, or an image reconstructor may be provided as a separate processing unit. Control system 112 provides control commands for operating gantry 102, an x-ray tube 114, a gantry motor controller 116, as examples. Gantry 102 includes a cover or enclosure 118, which provides for aesthetic improvement, safety, etc.

Gantry 102 includes a rotatable base 120, on which is mounted x-ray tube 114, a heat exchanger 122, a data acquisition system (DAS) 124, an inverter 126, a generator 128, and a detector assembly 130, as examples. System 100 is operated with commands entered by a user into computer 110. Gantry 102 may include gantry controls 132 located thereon, for convenient user operation of some of the commands for system 100. Detector assembly 130 includes a plurality of detector modules (e.g., a dual layer detector module 400), which include an anti-scatter grid (ASG; e.g., ASG 440), scintillators (e.g., a low-density scintillator 452, a high-density scintillator 462, etc.), photodiodes (e.g., a front-illuminated photodiode 454, a back-illuminated photodiode 464, etc.), and the like, which detect x-rays and convert the x-rays to electrical signals, from which imaging data is generated. Gantry 102 includes a pre-patient collimator 134 that is positioned to define or shape an x-ray beam 136 emitted from x-ray tube 114. Although not shown, a shape filter may be positioned for instance between x-ray tube 114 and pre-patient collimator 134.

In operation, rotatable base 120 is caused to rotate about the patient up to typically a few Hz in rotational speed, and table 106 is caused to move the patient axially within opening 104. When a desired imaging location of the patient is proximate an axial location where x-ray beam 136 will be caused to emit, x-ray tube 114 is energized and x-ray beam 136 is generated from a focal spot within x-ray tube 114. The detectors receive x-rays, some of which have passed through the patient, yielding analog electrical signals are digitized and passed to DAS 124, and then to computer 110 where the data is further processed to generate an image. The imaging data may be stored on computer system 100 and images may be viewed. An X-Y-Z triad 138, corresponding to a local reference frame for components that rotate on rotatable base 120, defines a local directional coordinate system in a gantry circumferential direction X, a gantry radial direction Y, and gantry axial direction Z. Accordingly, and referring to triad 138, the patient passes parallel to the Z-axis, the x-rays pass along the Y axis, and the rotational components (such as detector assembly 130) rotate in a circumferential direction and in the X direction, and about an isocenter 140 (which is a centerpoint about which rotatable base rotates, and is an approximate position of the patient for imaging purposes). A focal spot 142 is illustrated within x-ray tube 114, which corresponds to a spot from which x-ray beam 136 emits.

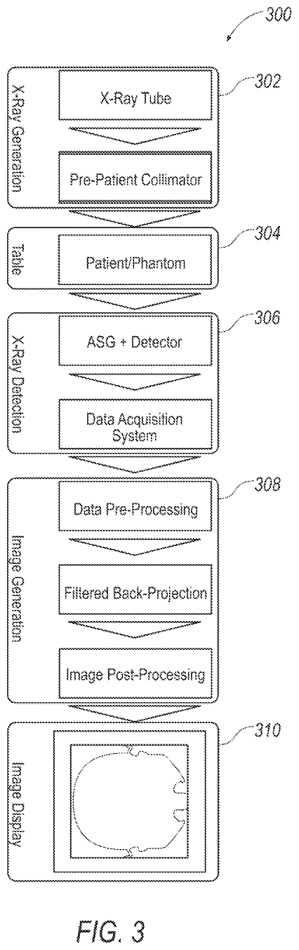

FIG. 3 illustrates an exemplary image chain 300, consistent with the operation described with respect to FIGS. 1 and 2. X-ray generation 302 occurs, using x-ray tube 114 and passing x-rays through pre-patient collimator 134, during which time table 106 passes 304 through opening 104 of gantry 102. In one example table 106 may have a patient thereon, and in another example a phantom may be used for calibration purposes.

X-ray detection 306 occurs when x-rays having emitted from x-ray tube 114 pass to detector assembly 130. An anti-scatter grid (e.g., ASG 440) prevents x-ray scatter (emitting for example from the patient as secondary x-rays and in a direction that is oblique to x-ray beam 136), by generally passing x-rays that emit from x-ray tube 114. DAS 124 processes signals received from detector assembly 130. Image generation 308 occurs after the digitized signals are passed from a rotating side of gantry 102 (on rotatable base 120) to a stationary side, via for instance a slipring.

Image generation 308 occurs in computer system 110, or in a separate processing module that is in communication with computer system 110. The data is pre-processed, and image views or projections are used to reconstruct images using known techniques such as a filtered backprojection (FBP). Image post-processing also occurs, after which the images may be displayed 310, or otherwise made available for display elsewhere (such as in a remote computing device).

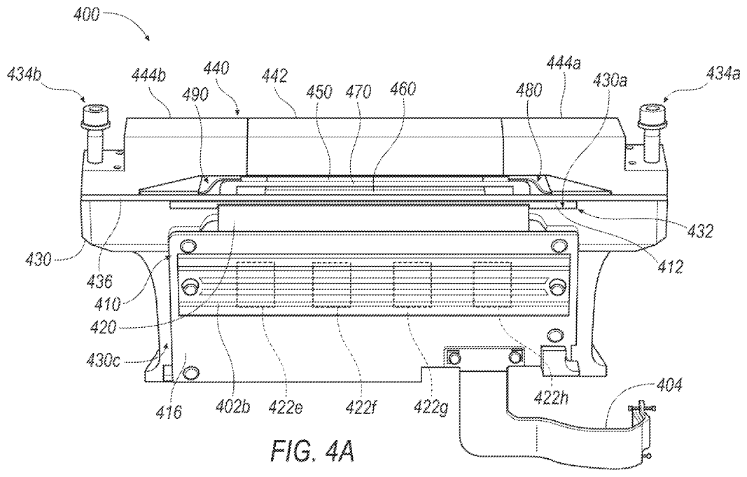

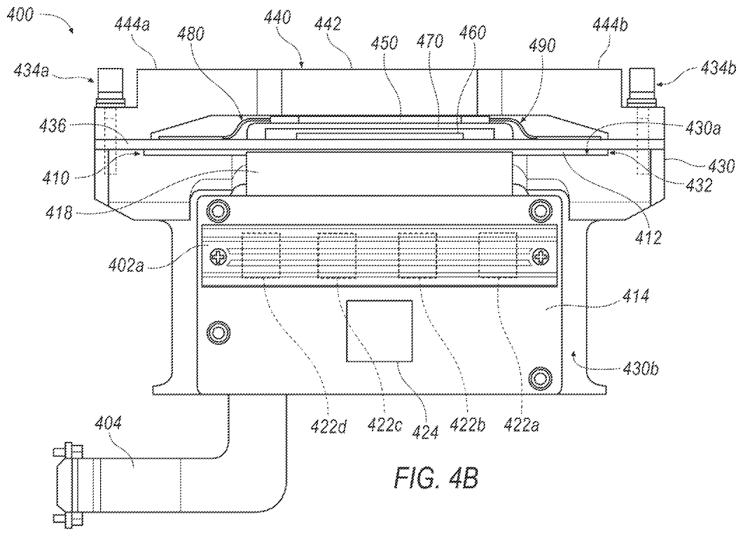

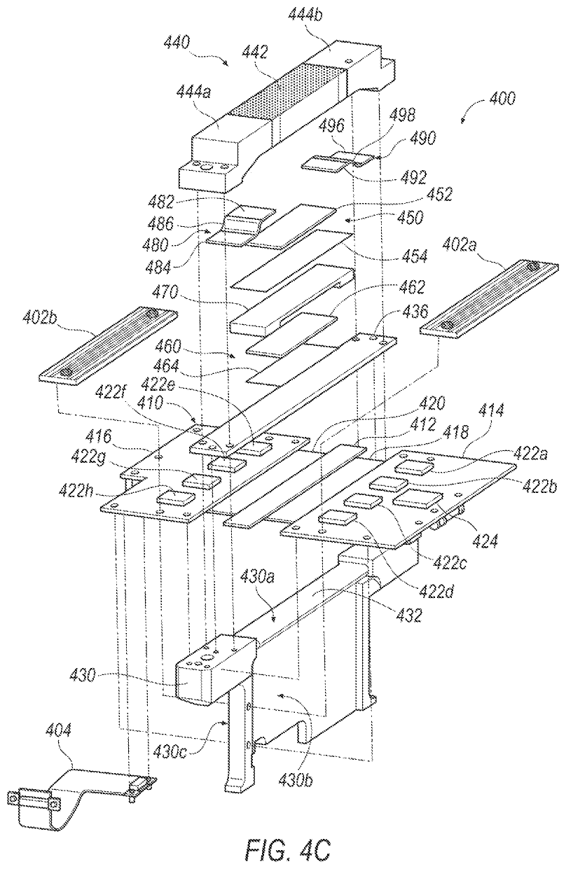

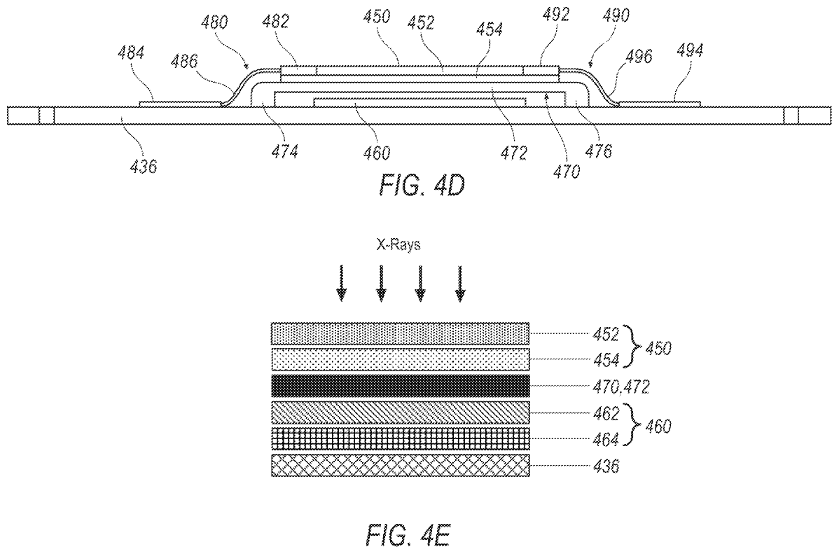

As generally shown in FIGS. 4A-8B, a dual layer detector module 400 includes one or more heat sinks 402a, 402b, a cable-connector 404, a first rigid flex board 410, a module frame 430, an anti-scatter grid (ASG) 440, a low-density scintillator-photodiode array 450, and a high-density scintillator-photodiode array 460. An alignment block or support structure may mechanically support the dual layer detector module 400. The dual layer detector module 400 may be arranged on a gantry of a CT system, such as system 100 above, and/or may have an orientation of a Z-direction (e.g., a slice direction) and an X-direction (e.g., a channel direction). The heat sinks 402a, 402b may be in thermal contact with the first rigid flex board 410 and/or one or more components arranged thereon for providing enhanced cooling to electronics arranged on the first rigid flex board 410. For example, a first heat sink 402a may be arranged on a first lateral portion 414 of the first rigid flex board 410 and contact and/or cover ASICs 422a-422d. A second heat sink 402b may be arranged on a second lateral portion 416 of the first rigid flex board 410 and contact and/or cover ASICs 422e-422h. One end of the cable-connector 404 is connected to the first rigid flex board 410 and a second, opposite end of the cable-connector 404 is operatively connected to the computer system 110 and/or the control system 112 such that the cable-connector 404 facilitates routing of signals, currents, information, etc. from the dual layer detector module 400 to the computer system 110 and/or the control system 112. While the cable-connector 404 is connected to the second lateral portion 416 of the first rigid flex board 410 in FIGS. 4A-4E, the cable-connector 404 may alternatively be connected to the first lateral portion 414 of the first rigid flex board 410.

The first rigid flex board 410 includes one or more conductors disposed on, connected to, and/or integrated therein via which a first rigid flex board 410 may facilitate transmission of and/or convey electrical signals and/or currents between two or more components, elements, structures, etc. The first rigid flex board 410 includes one or more electronic components for signal processing, wherein analog electrical signals (e.g., from one or more scintillator-photodiode arrays 450, 460) are digitized and then passed to DAS 124. The first rigid flex board 410 includes a central portion 412, a first lateral portion 414, a second lateral portion 416, a first connector portion 418, and a second connector portion 420. The central portion 412, the first lateral portion 414, and the second lateral portion 416 are each configured as a circuit board (e.g., a printed circuit board). The first lateral portion 414 and the second lateral portion 416 of the first rigid flex board 410 are disposed on opposite sides of the central portion 412 relative to one another. The first lateral portion 414 and the second lateral portion 416 are connected to the central portion 412 via the first connector portion 418 and the second connector portion 420, respectively. The first connector portion 418 and the second connector portion 420 are configured to transmit and/or convey electrical signals and/or currents between the central portion 412 and the associated lateral portion 414, 416.

The first connector portion 418 and the second connector portion 420 are each configured as a flexible portion (e.g., a first flexible portion, a second flexible portion), which may allow a position and/or orientation of the lateral portion(s) 414, 416 to be adjusted relative to the central portion 412. The first flexible portion 418 and the second flexible portion 420 are each configured as a high-density flex portion.

Additionally and/or alternatively, the first connector portion 418 and/or the second connector portion 420 may be configured as a curved and/or bent portion (e.g., a first bent portion, a second bent portion). The first bent portion and/or the second bent portion may be bent and/or curved such that the associated lateral portion 414, 416 is disposed transversely, obliquely, and/or perpendicularly relative to the central portion 412 (e.g., at a 90° angle).

One or more electrical connectors, circuit boards, electronics packages, processors, analog to digital ASICs (application-specific integrated circuit) or FPGA (field-programmable gate array), and/or other associated electronic components may be disposed on and connected to the first rigid flex board 410. For example, as illustrated in FIGS. 4A-4C, four A/D ASICs 422a-422d and one FPGA 424 are disposed on the first lateral portion 414 and four other A/D ASICs 422e-422h are disposed on the second lateral portion 416 of the first rigid flex board 410. In other examples, the FPGA 424 may be arranged on the second lateral portion 416 and/or on the same lateral portion 414, 416 to which the cable-connector 404 is connected.

The first rigid flex board 410 is disposed and/or mounted on and connected to the module frame 430. The central portion 412 of the first rigid flex board 410 is disposed on and/or aligned with a first surface 430a of the module frame 430. The module frame 430 includes a receptacle 432 (e.g., a recess, depression, notch, etc.), a bottom surface of which may be the first surface 430a. The central portion 412 of the first rigid flex board 412 is configured to be received at least partially within the receptacle 432. The first and/or second lateral portions 414, 416 of the first rigid flex board 410 are disposed on and connected to one or more surfaces 430b, 430c of the module frame 430. For example, the first lateral portion 414 is disposed on a second surface 430b of the module frame 430 (e.g., a first lateral surface 430b extending transversely to the first surface 430a), and the second lateral portion 416 is disposed on a third surface 430c of the module frame 430 (e.g., a second lateral surface 430c extending transversely to the first surface 430a and/or parallel to the second surface 430b). The connector portions 418, 420 enable such an arrangement. For example, the first flexible portion and the second flexible portion flex and/or deform to conform the first rigid flex board 410 to the shape of the module frame 430 when mounting the first rigid flex board 410. The first rigid flex board 410 and/or one or more portions thereof (e.g., portions 412, 414, 416) are connected to the module frame 430 via screws, but may be connected in a variety of manners such as with connectors, fasteners, pins, adhesive, chemical bonding, molding, etc.

The main routing substrate 436 is disposed on and connected to the first rigid flex board 410 (e.g., the central portion 412) and/or the module frame 430. The main routing substrate may include a multi-layer ceramic routing substrate. The main routing substrate 436 is to receive, gather, collect, assemble, merge, etc. one or more signals and/or currents from the low-density scintillator-photodiode array 450 and the high-density scintillator-photodiode array 460 (e.g., directly and/or via a thru-via substrate 502, one or more rigid flex boards 480, 490, 610, a high-density flex, etc.), which signals may convey information and/or data collected via the received x-rays. The main routing substrate 436 may be further configured to route the signals, currents, information, etc. to the first rigid flex board 410.

The ASG 440 has a plurality of plates 442 and two end blocks 444a, 444b. The two end blocks 444a, 444b are disposed on and connected to opposite sides of the plates 442. The plates 442 are oriented approximately parallel to a Y-Z plane of the detector assembly 130. The two end blocks 444a, 444b are each connected to the main routing substrate 436 such that the plates 442 are disposed above and aligned with the low-density scintillator-photodiode array 450 and the high-density scintillator-photodiode array 460. The ASG 440 and the main routing substrate 436 are connected to the module frame 430 via mounting screws 434a, 434b that engage the module frame 430 and the two end blocks 444a, 444b.

The low-density scintillator-photodiode array 450 is configured to collect low-energy data from received x-rays and the high-density scintillator-photodiode array 460 is configured to collect high-energy data from received x-rays. The low-density scintillator-photodiode array 450 is disposed above and aligned with the high-density scintillator-photodiode array 460 such that, during operation, x-rays interact with the low-density scintillator-photodiode array 450 prior to interacting with the high-density scintillator-photodiode array 460. The low-density scintillator-photodiode array 450 and the high-density scintillator-photodiode array 460 may collectively form/define a stack of multiple scintillator-photodiode arrays. The scintillator-photodiode arrays 450, 460 may each be arranged on a respective base substrate that may include a ceramic or other solid base material. The low-density scintillator-photodiode array 450 includes a low-density scintillator 452 (e.g., an Yttrium Aluminum Garnet or YAG scintillator) disposed on a front-illuminated first photodiode 454. The low-density scintillator 452 is disposed on a side of the first photodiode 454 opposite the main routing substrate 436. The high-density scintillator-photodiode array 460 includes a high-density scintillator 462 (e.g., Gadolinium OxySulfide or GOS scintillator) disposed on a back-illuminated second photodiode 464. The high-density scintillator 462 is disposed on a side of the second photodiode 464 opposite the main routing substrate 436. The first and second photodiodes 454, 464 are each optically coupled via an optical coupler to the associated scintillator 452, 462. The first and second scintillators 452, 462 may be pixelated scintillators. The first and second scintillators 452, 462 each include a plurality of pixels (e.g., an array of pixels), which may extend generally in the X-direction. The first and second photodiodes 454, 464 may be pixelated photodiodes. The first and second photodiodes 454, 464 each include a plurality of photodiode pixels (e.g., an array of pixels), which correspond with the pixels of the associated scintillator 452, 462.

The scintillator-photodiode arrays 450, 460 are physically, electrically, and/or communicatively connected to the main routing substrate 436. The scintillator-photodiode arrays 450, 460 may be connected to the main routing substrate 436 in a variety of different manners illustrative examples of which are shown in FIGS. 4A-8B.

In the illustrative example generally shown in FIGS. 4A-4E, the dual layer detector module 400 includes a mechanical substrate 470, a second rigid flex board 480, and a third rigid flex board 490. The high-density scintillator-photodiode array 460 is directly connected to the main routing substrate 436 both physically and electrically via a conductive epoxy ball array. The high-density scintillator 462 is arranged on the second photodiode 464, which is arranged on the main routing substrate 436 (i.e., the second photodiode 464 is arranged between the high-density scintillator 462 and the main routing substrate 436). The mechanical substrate 470 has a main section 472 and two leg sections 474, 476 protruding therefrom to provide the mechanical substrate 470 with a generally U-shaped profile. The two leg sections 474, 476 are each connected to the main routing substrate 436 and suspend the main section 472 over the high-density scintillator-photodiode array 460. The low-density scintillator-photodiode array 450 is disposed on and connected to the main section 472 of the mechanical substrate 470, which physically connects the low-density scintillator-photodiode array 450 to the main routing substrate 436. The low-density scintillator 452 is arranged on the first photodiode 454, which is arranged on the mechanical substrate 470 (i.e., the first photodiode 454 is arranged between the low-density scintillator 452 and the mechanical substrate 470). The second rigid flex board 480 includes a first board portion 482, a second board portion 484, and a connector portion 486. The first and second board portions 482, 484 are each configured as a circuit board (e.g., a printed circuit board) and include one or more conductors disposed on, connected to, and/or integrated therein, which may facilitate transmission of and/or convey electrical signals and/or currents between two or more components, elements, structures, etc. The connector portion 486 is configured as a flexible, high-density flex portion that physically and electrically connects the first and second board portions 482, 484. The third rigid flex board 490 includes a third board portion 492, a fourth board portion 494, and a connector portion 496 that are configured similarly to the corresponding features of the second rigid flex board 480 described above. The first board portion 482 is arranged on and electrically connected to the first photodiode 454 at a first end of the low-density scintillator-photodiode array 450. The second board portion 484 is arranged on and physically and electrically connected to the main routing substrate 436. The third board portion 492 is arranged on and electrically connected to the first photodiode 454 at a second end of the low-density scintillator-photodiode array 450 opposite the first end. The fourth board portion 494 is arranged on and physically and electrically connected to the main routing substrate 436. In this manner, the second and third rigid flex boards 480, 490 electrically connect the low-density scintillator-photodiode array 450 and the main routing substrate 436.

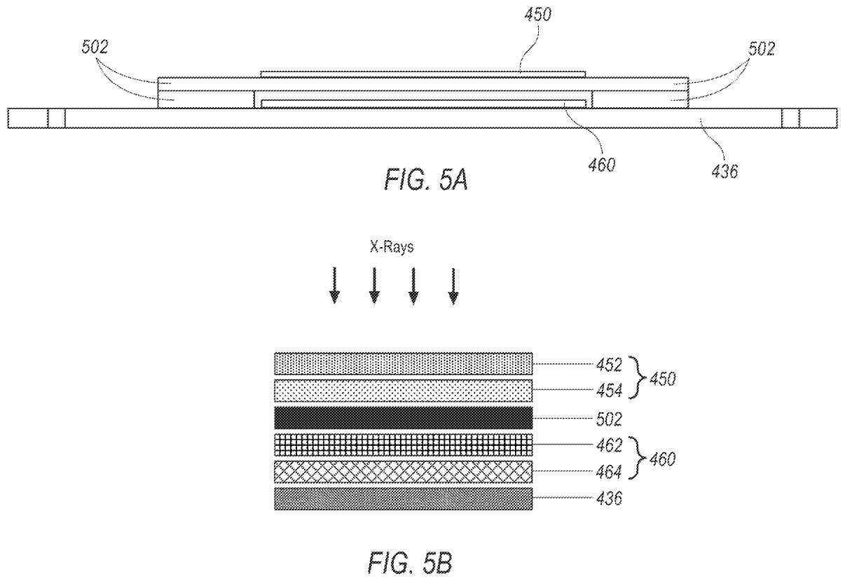

In the illustrative example generally shown in FIGS. 5A and 5B, the dual layer detector module 400 includes a thru-via substrate 502. The high-density scintillator-photodiode array 460 is structured and arranged as described above with respect to FIGS. 4A-4E. The low-density scintillator-photodiode array 450 is disposed on and connected to the thru-via substrate 502, which physically and electrically connects the low-density scintillator-photodiode array 450 to the main routing substrate 436. The low-density scintillator 452 is arranged on the first photodiode 454, which is arranged on the thru-via substrate 502 (i.e., the first photodiode 454 is arranged between the low-density scintillator 452 and the thru-via substrate 502). The arrangement of the components, elements, and/or features described above is generally illustrated in FIG. 5B as a simplified schematical representation with reference to x-rays that are to be received by the dual layer detector module 400 during operation.

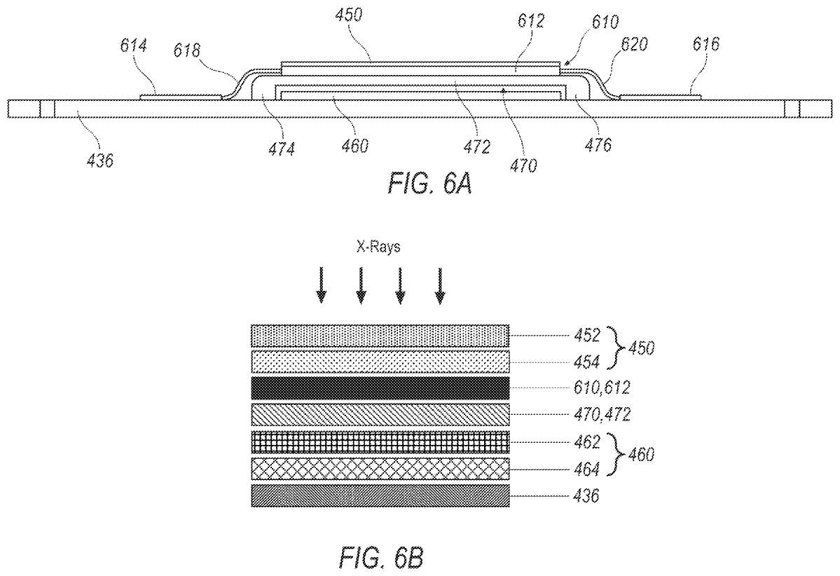

In the illustrative example generally shown in FIGS. 6A and 6B, the dual layer detector module 400 includes a fourth rigid flex board 610. The fourth rigid flex board 610 includes a central portion 612, a first lateral portion 614, a second lateral portion 616, a first connector portion 618, and a second connector portion 620 that are configured similarly to and/or in the same manner as the corresponding features of the first rigid flex board 410 described above. The high-density scintillator-photodiode array 460 and the mechanical substrate 470 are structured and arranged as described above with respect to FIGS. 4A-4E. The central portion 612 of the fourth rigid flex board 610 is disposed on and connected to the mechanical substrate 470. The first and second lateral portions 614, 616 of the fourth rigid flex board 610 are physically and electrically connected to the main routing substrate 436. The low-density scintillator-photodiode array 450 is disposed on and physically and electrically connected to the central portion 612. The low-density scintillator 452 is arranged on the first photodiode 454, which is arranged on the central portion 612 of the fourth rigid flex board 610 (i.e., the first photodiode 454 is arranged between the low-density scintillator 452 and the fourth rigid flex board 610). In this manner, the fourth rigid flex board 610 electrically connects the low-density scintillator-photodiode array 450 and the main routing substrate 436. The arrangement of the components, elements, and/or features described above is generally illustrated in FIG. 6B as a simplified schematical representation with reference to x-rays that are to be received by the dual layer detector module 400 during operation.

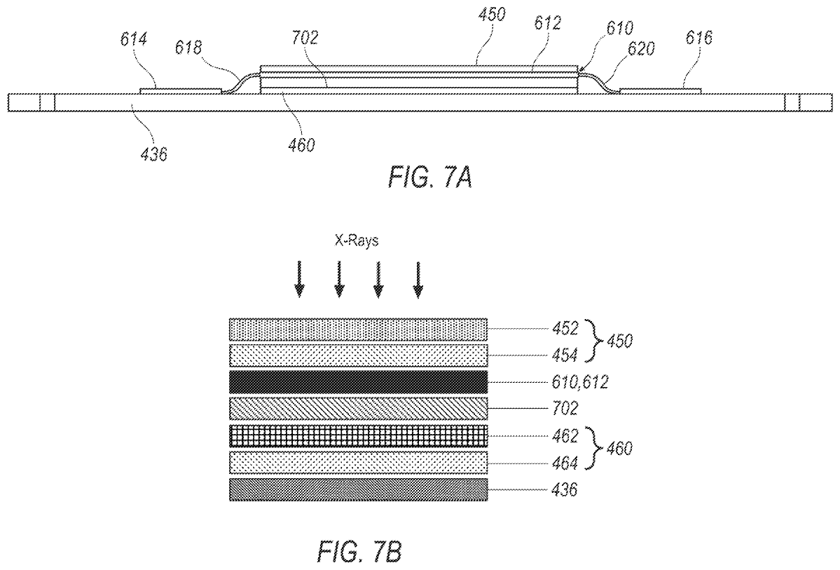

In the illustrative example generally shown in FIGS. 7A and 7B, the dual layer detector module 400 includes a filter 702 (e.g., an x-ray filter, a metallic x-ray filter, etc.). The high-density scintillator-photodiode array 460 is structured and arranged as described above with respect to FIGS. 4A-4E. The filter 702 is disposed on and connected to the high-density scintillator-photodiode array 460 opposite the main routing substrate 436 (i.e., the filter 702 is arranged on the high-density scintillator 462, which is arranged on the second photodiode 464, which is arranged on the main routing substrate 436). The central portion 612 of the fourth rigid flex board 610 is disposed on and connected to the filter 702 opposite the high-density scintillator-photodiode array 460. The first and second lateral portions 614, 616 of the fourth rigid flex board 610 are physically and electrically connected to the main routing substrate 436. The low-density scintillator-photodiode array 450 is disposed on and physically and electrically connected to the central portion 612 (i.e., the low-density scintillator 452 is arranged on the first photodiode 454, which is arranged on the central portion 612 of the fourth rigid flex board 610) such that the filter 702 and the fourth rigid flex board 610 are disposed between the low-density and high-density scintillator-photodiode arrays 450, 460. In this manner, the fourth rigid flex board 610 electrically connects the low-density scintillator-photodiode array 450 and the main routing substrate 436. Additionally, the arrangement of the filter 702 between the low-density and high-density scintillator-photodiode arrays 450, 460 improves energy separation between the high-density scintillator-photodiode array 460 and the low-density scintillator-photodiode array 450. The arrangement of the components, elements, and/or features described above is generally illustrated in FIG. 7B as a simplified schematical representation with reference to x-rays that are to be received by the dual layer detector module 400 during operation.

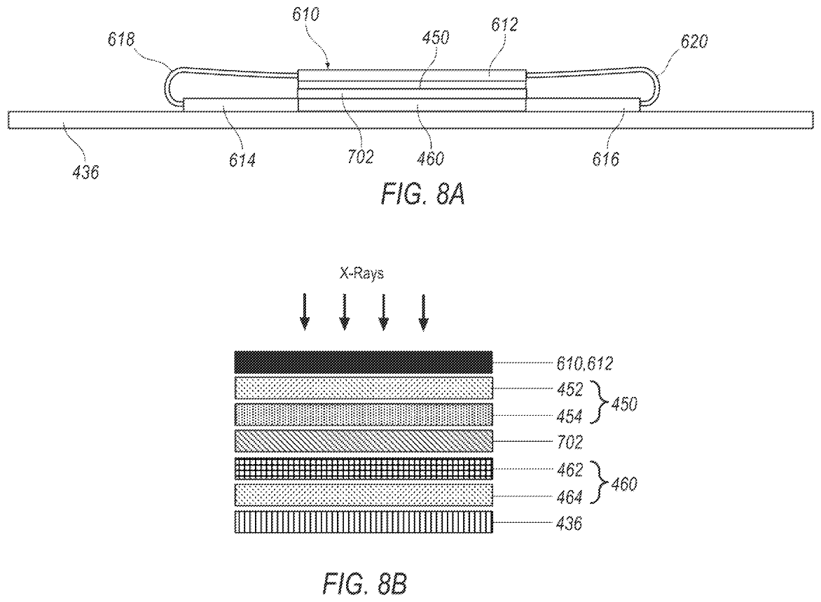

In the illustrative example generally shown in FIGS. 8A and 8B, the high-density scintillator-photodiode array 460 and the filter 702 are structured and arranged as described above with respect to FIGS. 7A and 7B. The low-density scintillator-photodiode array 450 is disposed on and connected to the filter 702 opposite the high-density scintillator-photodiode array 460 (i.e., the low-density scintillator 452 is arranged on the first photodiode 454, which is arranged on the filter 702, which is arranged on the high-density scintillator 462, which is arranged on the second photodiode 464). The central portion 612 of the fourth rigid flex board 610 is disposed on and physically and electrically connected to the low-density scintillator-photodiode array 450 (e.g., the low-density scintillator 452). The first and second lateral portions 614, 616 of the fourth rigid flex board 610 are physically and electrically connected to the main routing substrate 436. In this manner, the fourth rigid flex board 610 electrically connects the low-density scintillator-photodiode array 450 and the main routing substrate 436. Additionally, the arrangement of the filter 702 between the low-density and high-density scintillator-photodiode arrays 450, 460 improves energy separation between the high-density scintillator-photodiode array 460 and the low-density scintillator-photodiode array 450. The arrangement of the components, elements, and/or features described above is generally illustrated in FIG. 8B as a simplified schematical representation with reference to x-rays that are to be received by the dual layer detector module 400 during operation.

During use of the dual layer detector module 400 (e.g., during operation of the CT scanner), the low-density scintillator-photodiode array 450 receives and absorbs low-energy photons of the received x-rays to collect low-energy data, while allowing high-energy photons of the received x-rays to pass therethrough and reach the high-density scintillator-photodiode array 460, which receives and absorbs the high-energy photons of the received x-rays to collect high-energy data. The low-density scintillator-photodiode array 450 sends one or more signals and/or currents to the main routing substrate 436 via the second and third rigid flex boards 480, 490 (e.g., FIGS. 4A-4E), the thru-via substrate 502 (e.g., FIGS. 5A and 5B), and/or the fourth rigid flex boards 610 (e.g., FIGS. 7A-8A), which signals may convey the low-energy data collected via the received x-rays. In the example of FIGS. 4A-4E, a first subset of signals (e.g., half) are sent from the low-density scintillator-photodiode array 450 to the main routing substrate 436 by the first photodiode 454, the first board portion 482, the connector portion 486, and the second board portion 484. A second subset of signals (e.g., half) are sent from the low-density scintillator-photodiode array 450 to the main routing substrate 436 by the first photodiode 454, the third board portion 492, the connector portion 496, and the fourth board portion 494. In the examples of FIGS. 6A-8B, a first subset of signals (e.g., half) are sent from the low-density scintillator-photodiode array 450 to the main routing substrate 436 by the first photodiode 454, the central portion 612, the first connector portion 618, and the first lateral portion 614. A second subset of signals (e.g., half) are sent from the low-density scintillator-photodiode array 450 to the main routing substrate 436 by the first photodiode 454, the central portion 612, the second connector portion 620, and the second lateral portion 616. The high-density scintillator-photodiode array 460 sends one or more signals and/or currents directly to the main routing substrate 436, which signals may convey the high-energy data collected via the received x-rays. The main routing substrate 436 receives, gathers, collects, assembles, merges, etc. one or more signals and/or currents from the low-density scintillator-photodiode array 450 and/or the high-density scintillator-photodiode array 460, and then routes the signals, currents, information, etc. to the first rigid flex board 410, which in turn routes the signals, currents, information, etc. to the computer system 110 and/or the control system 112 via the cable-connector 404.

In examples, the scintillator-photodiode arrays 450, 460 may each have a 16×64 pixel array (i.e., 1024 pixels). In such a case, the connector portions 486, 496, 618, 620 may each be configured as 512 high-density flex to provide 1024 channels (i.e., one channel for each pixel) from the low-density scintillator-photodiode array 450 to the main routing substrate 436. Similarly, 1024 channels (i.e., one channel for each pixel) may also be provided from the high-density scintillator-photodiode array 460 to the main routing substrate 436 and, thus, a total of 2048 channels routed from the main routing substrate 436 to the first rigid flex board 410. Each ASIC 422a-422h may have 256 channels and, thus, 1024 channels are provided on each lateral portion 414, 416 of the first rigid flex board 410.

When introducing elements of various embodiments of the disclosed materials, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While the preceding discussion is generally provided in the context of medical imaging, it should be appreciated that the present techniques and processes are not limited to such medical contexts. The provision of examples and explanations in such a medical context is to facilitate explanation by providing instances of implementations and applications. The disclosed approaches may also be utilized in other contexts, such as the non-destructive inspection of manufactured parts or goods (i.e., quality control or quality review applications), and/or the non-invasive inspection or imaging techniques.

While the disclosed materials have been described in detail in connection with only a limited number of embodiments, it should be readily understood that the embodiments are not limited to such disclosed embodiments. Rather, that disclosed can be modified to incorporate any number of variations, alterations, substitutions or equivalent arrangements not heretofore described, but which are commensurate with the spirit and scope of the disclosed materials. Additionally, while various embodiments have been described, it is to be understood that disclosed aspects may include only some of the described embodiments. Accordingly, that disclosed is not to be seen as limited by the foregoing description, but is only limited by the scope of the appended claims.

What is claimed is:

1. A CT detector module, comprising:
a module frame;
a first rigid flex board;
a main routing substrate arranged on the first rigid flex board;
a high-density scintillator-photodiode array arranged on and electrically connected to the main routing substrate; and
a low-density scintillator-photodiode array electrically connected to the main routing substrate;
wherein the first rigid flex board includes a central portion, a first lateral portion, a second lateral portion, a first flexible portion extending between and connecting the central portion and the first lateral portion, and a second flexible portion extending between and connecting the central portion and the second lateral portion; and
wherein the central portion is arranged on a first surface of the mounting frame, the first lateral portion is disposed on a second surface of the mounting frame, and the second lateral portion is disposed on a third surface of the mounting frame.

2. The CT detector module of claim 1, wherein:
the low-density scintillator-photodiode array and the high-density scintillator-photodiode array are at least partially aligned with one another such that, during operation, x-rays interact with the low-density scintillator-photodiode array prior to interacting with the high-density scintillator-photodiode array; and
the main routing substrate is configured to (i) receive a plurality of signals from at least one of the low-density scintillator-photodiode array and the high-density scintillator-photodiode array and (ii) route at least some of the plurality of signals to the first rigid flex board.

3. The CT detector module of claim 1, wherein:
the high-density scintillator-photodiode array includes a high-density scintillator and a back-illuminated photodiode;
the back-illuminated photodiode is arranged on the main routing substrate; and
the high-density scintillator is arranged on the back-illuminated photodiode opposite the mounting substrate.

4. The CT detector module of claim 3, wherein the high-density scintillator is a Gadolinium OxySulfide scintillator.

5. The CT detector module of claim 1, wherein:
the low-density scintillator-photodiode array includes a low-density scintillator and a front-illuminated photodiode;
the low-density scintillator is arranged on the front-illuminated photodiode; and
the front-illuminated photodiode is disposed between the low-density scintillator and the mounting substrate.

6. The CT detector module of claim 5, wherein the low-density scintillator is a Yttrium Aluminum Garnet scintillator.

7. The CT detector module of claim 1, wherein the high-density scintillator-photodiode array is arranged directly on the mounting substrate.

8. The CT detector module of claim 1, further comprising a thru-via substrate, wherein:
the thru-via substrate is arranged on the main routing substrate;
the low-density scintillator-photodiode array is arranged directly on the thru-via substrate; and
the low-density scintillator-photodiode array is electrically connected to the main routing substrate via the thru-via substrate.

9. The CT detector module of claim 8, wherein the high-density scintillator-photodiode array is arranged directly on the mounting substrate and disposed spaced apart from the thru-via substrate.

10. The CT detector module of claim 1, further comprising a mechanical substrate, wherein:
the mechanical substrate is arranged on the main routing substrate; and
the low-density scintillator-photodiode array is arranged on the mechanical substrate.

11. The CT detector module of claim 10, wherein the high-density scintillator-photodiode array is arranged directly on the mounting substrate and disposed spaced apart from the mechanical substrate.

12. The CT detector module of claim 11, wherein the mechanical substrate includes a main section and two leg sections protruding therefrom providing the mechanical substrate with a U-shaped profile.

13. The CT detector module of claim 10, further comprising a second rigid flex board and a third rigid flex board, wherein:
the second rigid flex board is arranged at least partially on the mechanical substrate and the main routing substrate;
the third rigid flex board is arranged at least partially on the mechanical substrate and the main routing substrate; and
the low-density scintillator-photodiode array is electrically connected to the main routing substrate via the second rigid flex board.

14. The CT detector module of claim 13, wherein:
the second rigid flex board includes a first board portion, a second board portion, and a connector portion extending between and connecting the first board portion and the second board portion;
the third rigid flex board includes a first board portion, a second board portion, and a connector portion extending between and connecting the first board portion and the second board portion;
the first board portion of the second rigid flex board and the first board portion of the third rigid flex board are arranged on the mechanical substrate on opposite sides of the low-density scintillator-photodiode array; and
the second board portion of the second rigid flex board and the second board portion of the third rigid flex board are arranged on the main routing substrate.

15. The CT detector module of claim 10, further comprising a second rigid flex board, wherein:
the second rigid flex board is arranged at least partially on the mechanical substrate and the main routing substrate; and
the low-density scintillator-photodiode array is electrically connected to the main routing substrate via the second rigid flex board.

16. The CT detector module of claim 15, wherein:
the second rigid flex board includes a central portion, a first lateral portion, a second lateral portion, a first flexible portion extending between and connecting the central portion and the first lateral portion, and a second flexible portion extending between and connecting the central portion and the second lateral portion;
the central portion of the second rigid flex board is arranged on the mechanical substrate;
the low-density scintillator-photodiode array is arranged on the central portion of the second rigid flex board opposite the mechanical substrate; and
the first lateral portion and the second lateral portion of the second rigid flex board are arranged on the main routing substrate.

17. The CT detector module of claim 1, further comprising a filter, wherein:
the high-density scintillator-photodiode array is arranged directly on the mounting substrate;
the filter is arranged on the high-density scintillator-photodiode array; and
the low-density scintillator-photodiode array is arranged on the filter opposite the high-density scintillator-photodiode array.

18. The CT detector module of claim 17, further comprising a second rigid flex board, wherein:
the second rigid flex board is arranged at least partially on the mechanical substrate and the main routing substrate; and
the low-density scintillator-photodiode array is electrically connected to the main routing substrate via the second rigid flex board.

19. The CT detector module of claim 18, wherein:
the second rigid flex board includes a central portion, a first lateral portion, a second lateral portion, a first flexible portion extending between and connecting the central portion and the first lateral portion, and a second flexible portion extending between and connecting the central portion and the second lateral portion;
the central portion of the second rigid flex board is arranged on the mechanical substrate;
the low-density scintillator-photodiode array is arranged on the central portion of the second rigid flex board opposite the mechanical substrate; and
the first lateral portion and the second lateral portion of the second rigid flex board are arranged on the main routing substrate.

20. The CT detector module of claim 1, further comprising a mechanical substrate, wherein:
the high-density scintillator-photodiode array is arranged directly on the mounting substrate;
the mechanical substrate is arranged on the high-density scintillator-photodiode array; and
the low-density scintillator-photodiode array is arranged on the mechanical substrate opposite the high-density scintillator-photodiode array.

21. The CT detector module of claim 20, further comprising a second rigid flex board via which the low-density scintillator-photodiode array is electrically connected to the main routing substrate, wherein:
the second rigid flex board includes a central portion, a first lateral portion, a second lateral portion, a first flexible portion extending between and connecting the central portion and the first lateral portion, and a second flexible portion extending between and connecting the central portion and the second lateral portion;
the central portion of the second rigid flex board is arranged on the low-density scintillator-photodiode array; and
the first lateral portion and the second lateral portion of the second rigid flex board are arranged on the main routing substrate.
